(19) United States
(12) Patent Application Publication
(10) Pub. No.: US 2002/0076412 A1
(43) Pub. Date: Jun. 20, 2002

(54) METHODS FOR MODULATING THE IMMUNE SYSTEM

(76) Inventors: LAWRENCE STEINMAN, PALO ALTO, CA (US); SCOTT ZAMVIL, BOSTON, MA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 08/484,409

(22) Filed: Jun. 7, 1995

Related U.S. Application Data
(63) Continuation-in-part of application No. 08/125,407, filed on Sep. 22, 1993, now abandoned.
Application No. 08/125,407 is a continuation-in-part of application No. 08/066,325, filed on May 21, 1993, now Pat. No. 5,667,967.
Application No. 08/066,325 is a continuation of application No. 07/877,444, filed on Apr. 30, 1992, now abandoned.
Application No. 07/877,444 is a continuation-in-part of application No. 07/517,245, filed on May 1, 1990, now abandoned.
Application No. 07/877,444 is a continuation-in-part of application No. PCT/US91/02991, filed on May 1, 1991.
Application No. 07/877,444 is a continuation-in-part of application No. 07/379,500, filed on Jul. 12, 1989, now abandoned.
Application No. 07/379,500 is a continuation-in-part of application No. 07/086,694, filed on Aug. 17, 1987, now abandoned.

(51) Int. Cl. A61K 38/00; A61K 39/38; A01N 25/00

(52) U.S. Cl. 424/184.1; 514/12; 514/14; 514/15; 514/903

(57) ABSTRACT

Methods for modulating the immune system of an animal, as well as tolerizing such an immune system through the administration of one or more polypeptides derived from human myelin basic protein (hMBP), are provided. Such polypeptides include residues 87-99 of hMBP, as well as residues His-Phe-Phe-Lys and/or Lys-Ile-Phe-Lys of hMBP.

### CROSS-REFERENCE TO RELATED APPLICATIONS

This application is a continuation-in-part of U.S. Ser. No. 08/125,407, filed Sep. 22, 1993, which is a continuation-in-part of U.S. Ser. No. 08/066,325, filed May 21, 1993, which is a file wrapper continuation of U.S. Ser. No. 07/877,444, filed Apr. 30, 1992, which is continuation-in-part of U.S. Ser. No. 07/517,245, filed May 1, 1990, and International Application Ser. No. PCT/US91/02991, filed May 1, 1991. This application is also a continuation-in-part of application Ser. No. 07/379,500, filed Jul. 12, 1989, which is a continuation-in-part of application Ser. No. 07/086,694, filed Aug. 17, 1987, the disclosures of all of which are specifically incorporated herein by reference.

### ACKNOWLEDGMENTS

This invention was supported in part by grants from the NIH. The U.S. Government may have rights in this invention.

### TECHNICAL FIELD

The subject invention relates generally to the treatment of diseases, particularly autoimmune diseases.

### BACKGROUND OF THE INVENTION

Autoimmune diseases are a result of a failure of the immune system to avoid recognition of self. The attack by the immune system of host cells can result in a large number of disorders, including such neural diseases as multiple sclerosis and myasthenia gravis diseases of the joints, such as rheumatoid arthritis, attacks on nucleic acids, as observed with systemic lupus erythematosus and such other diseases associated with various organs, as psoriasis, juvenile onset diabetes, Sjogren's disease, and thyroid disease. These diseases can have a variety of symptoms, which can vary from minor and irritating to life-threatening.

Despite the extensive research efforts that have been involved with elucidating the basis for these diseases, the diseases for the most part have been recalcitrant to an understanding of their etiology in the development of therapeutic modes. Many of the diseases are believed to be associated with lymphocytic involvement, which can result in attack and degradation of proteins, cytotoxicity, and the like.

In the case of cancer, tumor infiltrating lymphocytes (TIL) are believed to be part of the body's defense mechanism to destroy the tumor. Efforts have been made to expand T-cells found in tumor tissue and return the culture expanded cells to the host.

The complexity of the immune system has been a daunting barrier to an understanding of the autoimmune diseases and the immune response to neoproliferative diseases. In attempting to understand the mechanisms involved with the immunological response, there is substantial interest in understanding in what manner the system degenerates to attack self. By understanding the relationships between the components of the immune system, the manner in which the immune system distinguishes between self and non-self, and the components the immune system associated with a particular disease, ways may be developed to diagnose individuals who may be susceptible to autoimmune diseases and provide therapies to protect such susceptible individuals from autoimmune disease during its onset and during its progress or to treat individuals with specific T-cells.

Multiple sclerosis (MS) is an inflammatory disease of the central nervous system characterized by myelin destruction (McFarlin and McFarland, New Engl. J. Med. 307:1183 1251 (1982)). At the site of demyelination, depletion of oligodendroglia cells and proliferation of astrocytes is usually observed. Raine and Traugott, “Immunoregulatory Processes in Experimental Allergic Encephalomyelitis and Multiple Sclerosis,” Elsevier, N.Y., 151-212 (1984); Prineas and Wright, Lab. Invest 38:409-421 (1978). There is an accumulation of morphologically identifiable macrophages, plasma cells and T lymphocytes, characteristic of an inflammatory response in the brain. Prineas, Handbook of Clinical Neurology, 3, Elsevier, N.Y., (1985) pp. 213-257. MHC Class II, positive antigen presenting cells and activated T-cells secreting various cytokines are present. Woodroofe et al., J. Neurol. Sci. 74:135-152 (1986); Hafler and Weiner, Ann. Neurol. 22:89-93 (1987); Hafler and Weiner, Immunol. Rev. 100:307-332 (1987); Hoffman, J. Exp. Med. 170:607-612 (1989). Several lines of evidence suggest that T lymphocytes migrate from the peripheral blood through the CNS compartment and participate directly in the promotion of brain lesions. Hoffman et al., J. Immunol. 136:3239-3245 (1986); Traugott, J. Neuroimmunol. 4:201-221 (1985). In studies of MS plaque tissue with monoclonal antibodies, it has been shown that the majority of T-cells have the helper inducer CD4 positive phenotype. Sobel et al., J. Exp. Med. 167:1313-1322 (1988). Also, by restriction fragment length polymorphism analysis, T-cell receptor Va and Vβ genes have been shown to contribute to the genetic control of susceptibility to this disease. Beall et al., J. Neuroimmunol. 21:59-66 (1989); Seboun et al., Cell 57:1095-1100 (1989); Oksenberg et al., Proc. Natl. Acad. Sci. USA 86:988-992 (1989) describe the use of TIL cells in the treatment of tumors (Barth et al., J. Immunol. 144:1531 (1990)).

HLA-DR2Dw2 is associated with increased susceptibility to MS. Terasaki et al., Science 193:1245-1247 (1976). Susceptibility to MS has been associated with certain MHC Class II genes. Oksenberg and Steinman, Current Opinion in Immunology 2:619-621 (1990). At the cellular level, oligoclonality of T-cells has been described in the cerebrospinal fluid (CSF) of MS patients. Lee et al., Ann. Neurol. 29:33-40 (1991). Oksenberg et al., Nature 345:344-346 (1990) describes the use of PCR to amplify TCR Va sequences from transcripts derived from MS brain lesions. Wucherpfennig et al. Science 248:1016-1019 (1990) and Ota et al., Nature 346:183 (1990) report studies of T-cell clones in man that recognize myelin basic protein.

A definitive treatment for MS has not been established. Historically, corticosteroids and ACTH have been used to treat MS. Basically, these drugs reduce the inflammatory response by toxicity to lymphocytes. Recovery may be hastened from acute exacerbations, but these drugs do not prevent future attacks or prevent development of additional disabilities or chronic progression of MS (Carter and Rodriguez, Mayo Clinic Proc. 64:664, 1989; Weiner and Hafler, Ann. Neurol. 23:211, 1988). In addition, the substantial side effects of steroid treatments make these drugs undesirable for long-term use.

Other toxic compounds, such as azathioprine, a purine antagonist, cyclophosphamide, and cyclosporine have been used to treat symptoms of MS. Like corticosteroid treatment, these drugs are beneficial at most for a short term and are highly toxic. Side effects include increased malignancies, leukopenias, toxic hepatitis, gastrointestinal problems, hypertension, and nephrotoxicity (Mitchell, Cont. Clin. Neurol. 77:231, 1993; Weiner and Hafler, supra). Antibody based therapies directed toward T-cells, such as anti-CD4 antibodies, are currently under study for treatment of MS. However, these agents may cause deleterious side effects by immunocompromising the patient.

More recently, cytokines such as IFN-γ and IFN-β have been administered in attempts to alleviate the symptoms of MS. However, a pilot study involving IFN-γ was terminated because 7 of 18 patients treated with this drug experienced a clinical exacerbation within one month after initiation of treatment. Moreover, there was an increase in the specific response to MBP (Weiner and Hafler, supra).

Betaseron, a modified beta interferon, has recently been approved for use in MS patients. Although Betaseron treatment showed some improvement in exacerbation rates (Paty et al., Neurology 43:662, 1993), there was no difference in the rate of clinical deterioration between treated and control groups (IFNB MS Study Group, Neurology 43:655, 1993; Paty et al., supra). Side effects were commonly observed. The most frequent of such side effects were fever (40%-58% of patients), flu-like symptoms (76% of patients), chills (46% of patients), mylagias (41% of patients), and sweating (23% of patients). In addition, injection site reactions (85%), including inflammation, pain, hypersensitivity and necrosis, were common (IFNB MS Study Group, supra; Connelly, Annals of Pharm. 28:610, 1994).

In view of the problems associated with existing treatments of MS, there is a compelling need for improved treatments which are more effective and are not associated with such disadvantages. The present invention exploits the use of polypeptides which antagonize a T-cell response to human myelin basic protein to effectively treat MS, while providing other related advantages.

### SUMMARY OF THE INVENTION

Briefly stated, the present invention provides methods for treating multiple sclerosis, comprising administering to a patient a therapeutically effective amount of a pharmaceutical composition comprising one or more polypeptides as described herein in combination with a physiologically acceptable carrier or diluent. In addition, methods for modulating the immune system of an animal, as well as tolerizing such an immune system through the administration of a pharmaceutical composition as described herein are provided.

In one aspect, the polypeptide consists of nine amino acids substantially homologous with residues 87-99 of human myelin basic protein (“hMBP”). Within a related aspect, the polypeptide comprises amino acid residues 87-99 of hMBP or residues substantially homologous therewith, wherein the polypeptide is about 15 amino acids. Within one embodiment of this aspect, at least one or at least two residues within the region 87-99 are substituted with other amino acids.

Within another aspect the polypeptide comprises amino acid residues His-Phe-Phe-Lys (Seq. ID No. 15) of Lys-Ile-Phe-Lys (Seq. ID No. 16) of hMBP, wherein the polypeptide is about 15 amino acids. Such a polypeptide may be used alone or joined by chemical means with another epitope of hMBP. Within a preferred embodiment, the polypeptide comprises both amino acid residues His-Phe-Phe-Lys (Seq. ID No. 15) and Lys-IIe-Phe-Lys (Seq. ID No. 16) of hMBP.

Still further aspects of the present invention provide pharmaceutical compositions comprising a polypeptide as described herein in combination with a physiologically acceptable carrier or diluent.

These and other aspects will become evident upon reference to the following detailed description and attached drawing. In addition, various references are set forth below which describe in more detail certain procedures or compositions. Each of these references are incorporated by reference in their entirety as if each were individually noted for incorporation.

### BRIEF DESCRIPTION OF THE DRAWINGS

FIG. 1 depicts the DNA and predicted amino acid sequence for human myelin basic protein (Seq. ID Nos. 1 and (2 respectively).

### DETAILED DESCRIPTION OF THE INVENTION

Polypeptides or oligopeptides are provided herein which induce an autoimmune response to a self antigen, or a portion thereof, and are capable of binding to an MHC antigen of a host susceptible to the autoimmune disease. The compositions may be employed to enhance protection, by serving to tolerize the host and prevent immune attack against the endogenous protein or cell producing the endogenous protein. For toleration, the subject peptides may be used alone, or conjugated to carrier molecules, such as tetanus toxin, bovine serum albumin, or be linked to an innocuous immunogen to which the host has been previously immunized.

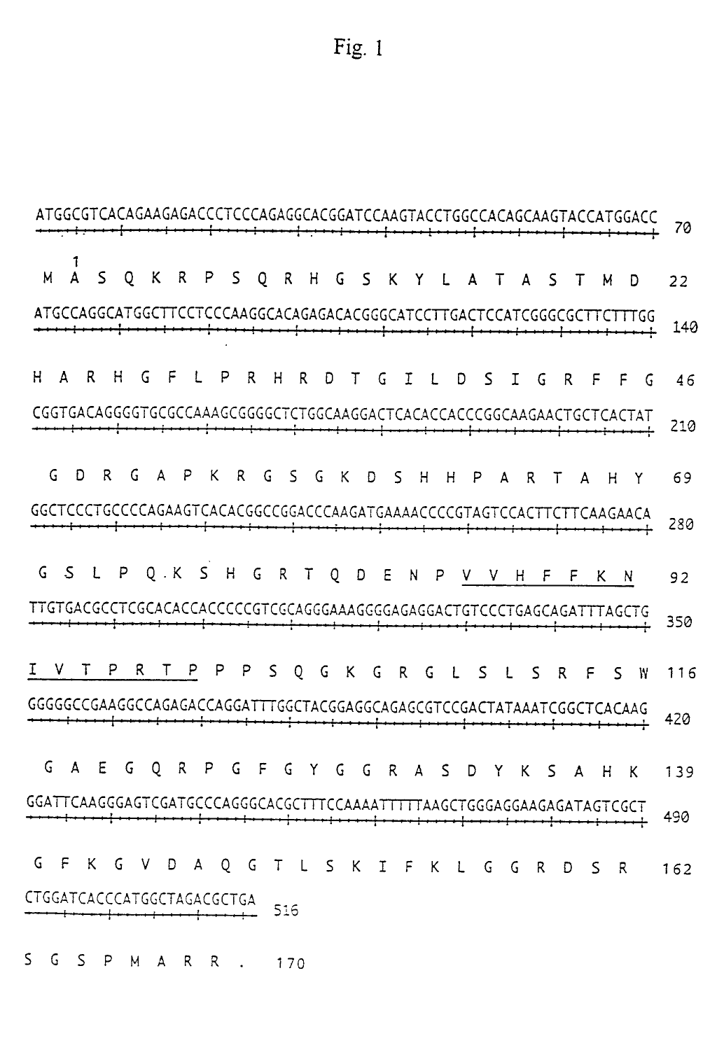

Sequences which may be employed for toleration will be sequences from proteins endogenous to the host involved with autoimmune diseases, such as multiple sclerosis, arthritis, diabetes, uveitis, and inflammatory bowel disease. With respect to multiple sclerosis, the subject invention provides polypeptides comprising amino acid residues of human myelin basic protein (“hMBP”), a protein found in the cytoplasm of human oligodendroglial cells. T-cell reactivity to autoantigens may be a critical component in the development of autoimmune diseases, such as T-cell responses against myelin basic protein, resulting in multiple sclerosis. The nucleotide sequence and predicted amino acid sequence of hMBP are presented in FIG. 1 (Seq. ID Nos. 1 and 2). Although not depicted in FIG. 1, different molecular forms of hMBP generated by differential splicing or post-translational modification are also within the scope of the present invention. Reference to particular residues of hMBP, such as “residue 91”(Lys) or “residues 87-99”(Val-Pro) refer to amino acids of hMBP as displayed in FIG. 1 or the amino acid(s) at a comparative position. More specifically, the numbering system for these residues relates to the amino acid position within the native human protein, regardless of the length of the peptide or the amino acid position within that peptide.

In general, the particular protein of interest will be screened for the presence of a subject motif, and one or more sequences including the motif selected. Where the histocompatibility genotype (haplotype) of the intended recipient is known, one sequence may be preferred over another. The polypeptides may be present as the individual peptides, or may be joined together in a single sequence, with or without intervening bridges, where any bridges will be other than the naturally occurring intervening sequences of the immunogen. Desirably, any such sequence would have fewer than about 100 amino acids, more usually fewer than about 60 amino acids. If there are a plurality of motifs present in the immunogen, all or fewer than all of the sequences including the motifs may be employed in a single composition.

The polypeptide comprising the subject motif may be from any site of the polypeptide sequence, that is N-terminal or C-terminal proximal or central, where the polypeptide sequence will normally be substantially homologous with from 9-15 amino acids of the immunogen sequence, although longer sequences (usually not more than 20-30 amino acids) may also be employed. Usually, the difference in homology between the natural sequence and the polypeptide which is employed will be not more than two alterations, more usually not more than 1 alteration, which may be insertions, deletions, conservative (similar charge, polarity, hydrophobicity, and bulkiness) or non-conservative substitutions. The composition may comprise one or more different polypeptides and may be joined covalently to other organic molecules, either proteinaceous or non-proteinaceous.

Usually, the motif sequence present in the polypeptide will be at other than the C-terminus of the polypeptide, desirably being at the N-terminus and not closer to the C-terminus than the center of the sequence. The N-terminal amino acid may be the same as the inducing peptide or may have an internal amino acid of the inducing peptide as the N-terminal amino acid of the polypeptide.

Within certain embodiments, the polypeptide sequence may be distinguished from the natural sequence. In some cases sequence analogs will be prepared with stepwise substitution of the amino acids with alanine or valine, particularly alanine. Each of the polypeptides may then be tested for their binding affinity to a host Class II MHC associated with restriction of T-cells involved with the autoimmune disease. Once a substitution has been identified as altering MHC antigen binding affinity, the same site may be further substituted with other amino acids to determine whether further alteration of MHC binding affinity may be achieved. Amino acids associated with T-cell recognition may also be substituted to diminish T-cell stimulation. Thus, those amino acids of the polypeptide associated with T-cell recognition may be modified to reduce T-cell stimulation in vivo, while not significantly affecting MHC antigen binding. In this way a strong blocking polypeptide may be achieved, without inducing the autoimmune action of the T-cells. Generally the total number of amino acids substituted will not exceed 2. An alternative polypeptide analog will have a functional group at the N-terminus, where the functional group would generally be from about 1 to 6, usually 1 to 3 carbon atoms.

Polypeptides within preferred embodiments of the present invention for use in treating multiple sclerosis should (a) compete for the binding of MBP to MHC; and (b) not cause proliferation of an MBP-reactive T-cell line. Candidate polypeptides may be screened for their ability to treat multiple sclerosis by (1) an assay measuring T-cell proliferation; (2) a competition assay; and (3) an assay assessing prevention of EAE. Those polypeptides that do not stimulate proliferation of MBP-reactive cell lines, inhibit the proliferation of MBP-reactive T-cells to native peptide and inhibit the development of EAE by native human MBP are useful therapeutics.

As noted above, candidate peptide analogues are first tested for their property of causing or inhibiting proliferation of T-cell lines. In the proliferation assay, MBP reactive T-cell lines may be used as target cells. T-cell lines are established from lymph nodes taken from rats injected with MBP. Lymph node cells are isolated and cultured for 5 to 8 days with MBP and IL-2 as a source of T-cell growth factors. Viable cells are recovered and a second round of stimulation is performed with MBP and irradiated splenocytes as a source of growth factors. After 5 to 6 passages in this manner, the proliferative potential of the cell lines are determined. MBP-reactive lines are used in the proliferation assay. In this assay, T-cell lines are cultured for three days with various concentrations of peptide analogues and irradiated, autologous splenocytes. After three days, 0.5-1.0 μCi of [³H]-thymidine is added for 12-16 hours. Cultures are harvested and incorporated counts determined. Mean CPM and standard error of the mean are calculated from triplicate cultures. Peptide analogues which do not stimulate proliferation at concentrations of less than or equal to 50 μM are suitable for further screenings.

The second assay is a competition assay for T-cell proliferation. In this assay, antigen presenting spleen cells are first irradiated and then incubated with native MBP peptide for 2-4 hours. These cells are then washed and further cultured with T-cells reactive to MBP. Various concentrations of candidate peptide analogues are included in cultures for an additional 3 days. Following this incubation period, each culture is pulsed with 1 μCi of [³H]-thymidine for an additional 12-18 hours. Cultures are then harvested on fiberglass filters and counted as above. Mean CPM and standard error of the mean are calculated from data determined in triplicate cultures. Peptide analogues which inhibit proliferation to approximately 25% at a concentration of 50 μM or greater are suitable for further screening.

Candidate peptides that do not cause direct proliferation of T-cell line or can inhibit proliferation by MBP, are further tested for their ability to inhibit the induction of EAE by MBP. Briefly, 500 μg of MBP is injected as an emulsion in complete Freund's adjuvant supplemented with heat killed Mycobacterium tuberculosis (H37Ra). Rats are injected subcutaneously at the base of the tail with 200 μl of the emulsion. Rats are divided into two groups. Approximately 2 days prior to disease induction (usually 10 days following injection of MBP) rats are injected intraperitoneally either with PBS or peptide analogues in PBS. Animals are monitored for clinical signs on a daily basis by an observer blind to the treatment protocol. EAE is scored on a scale of 0 4: 0, clinically normal; 1, flaccid tail paralysis; 2, hind limb weakness; 3, hind limb paralysis; 4, front and hind limbs affected. Peptide analogues injected at 5 mg/kg or less (approximately 1 mg per rat) are considered to inhibit the development of EAE if there is a 50% reduction in the mean cumulative score over seven days following onset of disease symptoms in the control group.

The following sequences (Seq. ID Nos. 3 to 38 respectively) may be used with advantage for tolerization by themselves or in conjunction with each other or other epitope sequences: G-A-P-S; G-A-V-G; E-W-V-S; K-V-P-T; G-V-V-L-G; G-A-V-I-G; G-I-L-G; K-A-A-S (associated with P₀); Ac-A-S-Q-K-R; K-Y-L-A-T; G-I-L-D; R-F-F-G; H-F-F-K; K-I-F-K (associated with P₁); Ac-S-N-K-F-L; K-F-L-G; K-L-V-S; E-Y-M-K; G-L-A-T; R-V-I-I-S; K-M-V-V-E; R-I-Y-E (associated with P₂); G-L-L-E; K-L-I-E; H-A-F-Q; G-A-V-R; K-W-L-G; K-F-V-G; R-M-Y-G; K-L-M-G (associated with PLP); and L-V-A-K; K-I-W-R; E-W-V-I-K; K-V-F-I-D; K-I-F-T; K-Y-I-A-E (associated with AChR). These sequences will normally be part of polypeptide sequences of about 9 to 15 amino acids as described for the other motifs.

The polypeptides may be prepared in a variety of ways, conveniently, in accordance with standard chemistry techniques, including synthesis by automated procedure. In general, peptide analogues are prepared by solid-phase peptide synthesis methodology which involves coupling each protected amino acid residue to a resin support, preferably a 4-methyl-benzhydrylamine resin, by activation with dicyclohexylcarbodimide to yield a peptide with a C-terminal amide. Alternatively, a chloromethyl resin (Merrifield resin) may be used to yield a peptide with a free carboxylic acid at the C-terminus. Side-chain functional groups are protected as follows: benzyl for serine, threonine, glutamic acid, and aspartic acid; tosyl for histidine and arginine; 2-chlorobenzyloxycarbonyl for lysine and 2,6-dichlorobenzyl for tyrosine. Following coupling, the t-butyloxycarbonyl protecting group on the alpha amino function of the added amino acid is removed by treatment with trifluoroacetic acid followed by neutralization with di-isopropyl-ethylamine. The next protected residue is then coupled onto the free amino group, propagating the peptide chain. After the last residue has been attached, the protected peptide-resin is treated with hydrogen fluoride to cleave the peptide from the resin, as well as deprotect the side chain functional groups. Crude product can be further purified by gel filtration, HPLC, partition chromatography, or ion-exchange chromatography.

Where larger sequences are involved, such as 30 amino acids or more, recombinant DNA techniques may be employed, where the gene may be synthesized in accordance with conventional ways, such as commercially available DNA synthesizers, expanded employing the polymerase chain reaction, and then inserted into an appropriate vector having the necessary transcriptional and translational initiation and termination regions. The resulting vector is then transformed into a host in which the expression vector is replicated and functional expression is obtained. The product may be secreted and harvested from the medium or when not secreted and retained cytoplasmically, the cells are harvested, lysed, and the desired protein isolated and purified in accordance with conventional ways.

Polypeptides of the present invention may be administered either alone, or as a pharmaceutical composition. Briefly, pharmaceutical compositions of the present invention may comprise one or more of the polypeptides described herein, in combination with one or more pharmaceutically or physiologically acceptable carriers, diluents or excipients. Such compositions may comprise buffers such as neutral buffered saline, phosphate buffered saline and the like, carbohydrates such as glucose, mannose, sucrose or dextrans, mannitol, proteins, polypeptides or amino acids such as glycine, antioxidants, chelating agents such as EDTA or glutathione, adjuvants (e.g., aluminum hydroxide) and preservatives. In addition, pharmaceutical compositions of the present invention may also contain one or more additional active ingredients, such as, for example, cytokines like β-interferon.

Compositions of the present invention may be formulated for the manner of administration indicated, including for example, for oral, nasal, venous, intracranial, intraperitoneal, subcutaneous, or intramuscular administration. Within other embodiments of the invention, the compositions described herein may be administered as part of a sustained release implant. Within yet other embodiments, compositions of the present invention may be formulized as a lyophilizate, utilizing appropriate excipients which provide stability as a lyophilizate, and subsequent to rehydration.

Pharmaceutical compositions of the present invention may be administered in a manner appropriate to the disease to be treated (or prevented). The quantity and frequency of administration will be determined by such factors as the condition of the patient, and the type and severity of the patient's disease. Within particularly preferred embodiments of the invention, the polypeptide or pharmaceutical compositions described herein may be administered at a dosage ranging from 0.01 to 100 mg/kg of host, more usually from about 1 to 10 mg/kg of host, where the concentration ranges from 5 to 50 mg/ml, although appropriate dosages may be determined by clinical trials. Patients may be monitored for therapeutic effectiveness by EDSS and signs of clinical exacerbation, as described above.

Treatment and Prevention of Multiple Sclerosis

As noted above, the present invention provides methods for treating and preventing multiple sclerosis by administering to the patient a therapeutically effective amount of a polypeptide of human myelin basic protein as described herein. Patients suitable for such treatment may be identified by criteria establishing a diagnosis of clinically definite MS as defined by the workshop on the diagnosis of MS (Poser et al., Ann. Neurol. 13:227, 1983). Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. Slightly lower criteria are used for a diagnosis of clinically probable MS.

Effective treatment of multiple sclerosis may be examined in several different ways. Satisfying any of the following criteria evidences effective treatment. Three main criteria are used: EDSS (extended disability status scale), appearance of exacerbations or MRI (magnetic resonance imaging).

The EDSS is a means to grade clinical impairment due to MS (Kurtzke, Neurology 33:1444, 1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step defines an effective treatment in the context of the present invention (Kurtzke, Ann. Neurol. 36:573-79, 1994).

Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are either mild, moderate, or severe according to changes in a Neurological Rating Scale (Sipe et al., Neurology 34:1368, 1984). An annual exacerbation rate and proportion of exacerbation-free patients are determined. Therapy is deemed to be effective if there is a statistically significant difference in the rate or proportion of exacerbation-free patients between the treated group and the placebo group for either of these measurements. In addition, time to first exacerbation and exacerbation duration and severity may also be measured. A measure of effectiveness as therapy in this regard is a statistically significant difference in the time to first exacerbation or duration and severity in the treated group compared to control group.

Candidate patients for prevention may be identified by the presence of genetic factors. For example, a majority of MS patients have HLA-type DR2a and DR2b. The MS patients having genetic dispositions to MS who are suitable for treatment fall within two groups. First are patients with early disease of the relapsing remitting type. Entry criteria would include disease duration of more than one year, EDSS score of 1.0 to 3.5, exacerbation rate of more than 0.5 per year, and free of clinical exacerbations for 2 months prior to study. The second group would include people with disease progression greater than 1.0 EDSS unit/year over the past two years.

The following examples are offered by way of illustration and not by way of limitation.

### EXAMPLES

### Example I

Prevention of EAE with Competitive Peptides

Synthetic MBP Peptides: Peptides corresponding to the amino acid sequences of rat (R) and bovine (B) MBP (Martenson, 1984, In Experimental Allergic Encephalomyelitis. A Useful Model for Multiple Sclerosis. Alvard, ed. Alan Liss, N.Y.), were synthesized using solid phase techniques (Erickson and Merrifield, 1976, in The Proteins, Vol. 2, Neurath, ed. Academic Press, NY, p. 255). Peptides were separated from the various organic side products and the purity was determined by high pressure liquid phase column (Merck, Darmstadt, Germany) and by amino acid analysis. These peptides were not further purified since they all contained greater than 90% of the desired product. For tolerization, the peptides may be used alone, conjugated to lymphocytes (Sriram, et al., 1983, supra) or coupled to a carrier such as tetanus toxoid or bovine serum albumin, employing conventional linking groups (Herzenberg, et al., Ann. Rev. Imm. 1:609-632, 1983).

Development of MBP-specific T-cell lines in the rat: T-cell lines are selected from LN or SC (spinal cords) of rats immunized with guinea pig myelin basic protein or with MBP peptide (200 μg) in CFA. Supernatants from ConA-stimulated Lewis rat splenocytes are used as the source of IL-2 to expand Ag-stimulated T-cells.

Proliferation Assay: Proliferative responses were determined as described previously (Zamvil, et al., Nature 317:355, 1985). 1×10⁴ T-cells were cultured with 5×10⁵ X-irradiated (3,000 rad) PL/J splenic APC in 0.2 ml of culture media in 96 well flat-bottomed microtiter plates (Falcon, 3072). Peptides were added to culture giving the final concentrations indicated. At 48 hours incubation, each well was pulsed with 1 μCi ³H-thymidine and harvested 16 hours later. The mean c.p.m. thymidine incorporation was calculated for triplicate cultures. Standard deviations from replicate cultures were within 10% mean value. Proliferation assays were performed in 96-well microtiter plates. Briefly, 2×10⁴ T-cells and 10⁶ irradiated thymocytes/well were incubated with stimulation medium only, Con A, or antigen. The cultures were harvested onto glass fiber filters and TdR uptake was assessed by liquid scintillation. Mean cpm were calculated from triplicate wells. In some experiments, competitor peptides, or anti I-A (OX-6) or anti-I-E antibodies (OX-17) were used to evaluate which MHC molecules were used to restrict the T-cell response, or to determine whether peptides could antagonize stimulation by the native peptide.

### Example II

Binding Specificity of Synthetic Peptides 87-99 of MBP

A set of substituted peptides based on the sequence VHFFKNIVTPRT (Seq. ID No. 39), which is identical in rats, mice and human myelin basic protein (MBP), and corresponds to the I-E restricted epitope MBP 87-99 in the rat was produced. The peptides are shown in Table 1 (Seq. ID Nos. 39 to 52 respectively) MHC binding was measure say described in Smilek et al, 1991, Gautam et al 1992a&b.

TABLE 1
Set of Alanine Substituted Peptides for Myelin Basic Protein Epitope
in Rat and Man

| | | | | | | | | | | | | |
|---|---|---|---|---|---|---|---|---|---|---|---|---|
| 87 | 88 | 89 | 90 | 91 | 92 | 93 | 94 | 95 | 96 | 97 | 98 | 99 |
| V | H | F | F | K | N | I | V | T | P | R | T | P |

| A | | ala |
|---|---|---|
| | | 1 |
| | A | ala |
| | | 2 |

| | |
|---|---|
| A | ala |
| | 3 |

| | |
|---|---|
| A | ala |
| | 4 |

| | |
|---|---|
| A | ala |
| | 5 |

| | |
|---|---|
| A | ala |
| | 6 |

| | |
|---|---|
| A | ala |
| | 7 |

| | |
|---|---|
| A | ala |
| | 8 |

| | |
|---|---|
| A | ala |
| | 9 |

| | |
|---|---|
| A | ala |
| | 10 |

| | |
|---|---|
| A | ala |
| | 11 |

| | |
|---|---|
| A | ala |
| | 12 |

| | |
|---|---|
| A | ala |
| | 13 |

The peptides were tested for their ability to cause EAE when mixed in CFA. As can be seen in Table 2, column 2, below, the peptides Ala4, Ala5, Ala6, Ala8, and Ala 10 were incapable of causing EAE, while Ala9 induced EAE in only 1/6 rats. It was seen that Ala4, Ala6, Ala7, and Ala8 are poor binders to I-E, implying that these residues 4F,6N,7I, and 8V are critical in MHC binding. The peptides Ala6 and Ala8 weakly stimulate an encephalitogenic T-cell line raised against native peptide (87-99). In the Lewis rat both Ala6 and Ala8 can block proliferation of an 87-99 T-cell line when given competitively with native peptide in vitro. Despite their inability to bind well to I-E, peptides ala6 and ala8 block the development of EAE when mixed with native peptide in a 5:1 molar ratio with CFA (0/6 sick with native plus ala6, 2/6 with native plus ala8, compared to 11/12 with native peptide alone). These competitor peptides Ala6 and Ala8, though poor I-E binders can apparently compete with native peptide and antagonize the T-cell receptor.

TABLE 2
Summary of Assays With Substituted Peptides In Rat #

| | | | (M*C) | | | Comp | |
|---|---|---|---|---|---|---|---|
| | | | IC50 | | | Assay | |
| | | EAE | I-E | | | Prolif | EAE |
| | peptide | (RAT) | Binding | Prolif. | CTL | (RAT) | (RAT) |
| | native | 21/21 | 14 μM | 4+ | nega- | MINUS | 11/12 |
| | | | | | tive | | |
| 87 | Ala 1 | 6/6 | 31 | 4+ | ND | ND | ND |
| V→A | | | | | | | |
| 88 | Ala 2 | 6/6 | 14 | 3+ | ND | ND | ND |
| H→A | | | | | | | |
| 89 | Ala 3 | 6/6 | 11 | 3+ | ND | ND | ND |
| F→A | | | | | | | |
| 90 | Ala 4 | 0/6 | >200 | negative | ± | ND | ND |
| F→A | | | | | | | |
| 91 | Ala 5 | 0/6 | 21 | negative | ND | ND | ND |
| K→A | | | | | | | |
| 92 | Ala 6 | 0/21 | >200 | 1+ | ND | plus | 0/6 |
| N→A | | | | | | | |
| 93 | Ala 7 | 6/6 | >200 | 2+ | plus | ND | ND |
| I→A | | | | | | | |
| 94 | Ala 8 | 1/15 | >200 | 1+ | plus | plus | 2/6 |
| V→A | | | | | | | |
| 95 | Ala 9 | 1/6 | 14 | negative | ND | ND | ND |
| T→A | | | | | | | |
| 96 | Ala 10 | 0/6 | 14 | 1+ | ND | ND | ND |
| P→A | | | | | | | |
| 97 | Ala 11 | 4/6 | 20 | negative | ND | ND | ND |
| R→A | | | | | | | |
| 98 | Ala 12 | 6/6 | 14 | 1+ | ND | ND | ND |
| T→A | | | | | | | |
| 99 | Ala 13 | 3/6 | 10 | 2+ | ND | ND | ND |
| P→A | | | | | | | |

Data shown above demonstrate formulations which are weak MHC binders which nevertheless antagonize TCR recognition of MBP peptide 87-99, and which prevent EAE when mixed with MBP in complete Freund's adjuvant in a 5:1 molar ratio. In addition Ala9 and Ala 10 are good MHC binders yet are nonencephalitogenic and are weak stimulators of an encephalitogenic T-cell line induced with native 87-99.

The compound Ala4 is a weaker binder to I-E (IC₅₀>200 mM), and does not stimulate an 87-99 T-cell line, and does not cause EAE. These compounds may antagonize TCR recognition of native 87-99 as well.

### Example III

Prevention of EAE with Synthetic Peptide 87-99 of MBP

In the following experiment, the possibility of preventing EAE by co-immunizing with MBP and a poor MHC binder, like Ala5 or Ala6, with the capacity to block a MBP response, is demonstrated.

TABLE 3
Competition with Ala6 and Ala5 For Prevention of EAE With MBP

| Native MBP | | | Incidence of |
|---|---|---|---|
| Peptide | ala 6 | ala 5 | EAE |
| 0.2 mg | | | 11/12 |
| | 0.2 mg | | 0/6 |
| | | 0.2 mg | 0/6 |
| 0.2 mg | | | 6/6 |
| 0.2 mg | 1 mg | | 0/6 |
| 0.2 mg | | 1 mg | 2/6 |

Incidence of EAE was expressed as number of mice with clinical EAE/number of mice immunized. For the induction of EAE, MBP peptide was dissolved in phosphate buffered saline (PBS) and emulsified with complete Freund's adjuvant (CFA) in a 1:1 mixture of PBS and CFA. Mice were injected with 0.2 ml emulsion at the base of the tail. On the same day and 48 h later, pertussis toxin (List Chemicals, Campbell, Calif.) was injected intravenously. Mice were examined daily for signs of EAE. For prevention of EAE, animals were immunized with MBP (0.2 mg), or in a mixture with the competitor peptide (1 mg).

As shown in Table 3, co-injection of Ala 6 completely prevented the clinical development of EAE. In addition, Ala 5 had a preventative effect on EAE.

It is evident from the above results, by modification of a peptide, particularly an internal peptide of a larger peptide that combines to an MHC and is associated with an autoimmune disease or other immune attack on mammalian cells, particularly syngeneic cells, the host may be protected from the immune attack. Thus, as T-cell immunodominant sequences are identified, these sequences may be modified by modifying the amino acid sequence to produce antagonists to the autoimmune disease.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of modulating the immune system of an animal, comprising administering to the animal a polypeptide consisting of nine amino acids substantially homologous with residues 87-99 of human myelin basic protein (hMBP).

2. A method of modulating the immune system of an animal, comprising administering to the animal a polypeptide comprising amino acid residues 87-99 of hMBP or residues substantially homologous therewith, wherein the polypeptide is about 15 amino acids.

3. A method of modulating the immune system of an animal, comprising administering to the animal a polypeptide comprising amino acid residues 87-99 of hMBP or residues substantially homologous therewith, wherein at least one residue within the region 87-99 is substituted with another amino acid.

4. A method of modulating the immune system of an animal, comprising administering to the animal a polypeptide comprising amino acid residues 87-99 of hMBP or residues substantially homologous therewith, wherein at least two residues within the region 87-99 are substituted with other amino acids.

5. A method of modulating the immune system of an animal, comprising administering to the animal a polypeptide comprising amino acid residues His-Phe-Phe-Lys of hMBP, wherein the polypeptide is about 15 amino acids.

6. A method of modulating the immune system of an animal, comprising administering to the animal a polypeptide comprising amino acid residues His-Phe-Phe-Lys of hMBP, joined by chemical means with another epitope of hMBP.

7. A method of modulating the immune system of an animal, comprising administering to the animal a polypeptide comprising amino acid residues Lys-Ile-Phe-Lys of hMBP, wherein the polypeptide is about 15 amino acids.

8. method of modulating the immune system of an animal, comprising administering to the animal a polypeptide comprising amino acid residues Lys-Ile-Phe-Lys of hMBP, joined by chemical means with another epitope of hMBP.

9. A method of modulating the immune system of an animal, comprising administering to the animal a polypeptide comprising amino acid residues His-Phe-Phe-Lys and Lys-Ile-Phe-Lys of hMBP.

10. A method of tolerizing the immune system of an animal, comprising administering to the animal a polypeptide consisting of nine amino acids substantially homologous with residues 87-99 of human myelin basic protein (hMBP).

11. A method of tolerizing the immune system of an animal, comprising administering to the animal a polypeptide comprising amino acid residues 87-99 of hMBP or residues substantially homologous therewith, wherein the polypeptide is about 15 amino acids.

12. A method of tolerizing the immune system of an animal, comprising administering to the animal a polypeptide comprising amino acid residues 87-99 of hMBP or residues substantially homologous therewith, wherein at least one residue within the region 87-99 is substituted with another amino acid.

13. A method of tolerizing the immune stem of an animal, comprising administering to the animal a polypeptide comprising amino acid residues 87-99 of hMBP or residues substantially homologous therewith, wherein at least two residues within the region 87-99 are substituted with other amino acids.

14. method of tolerizing the immune system of an animal, comprising administering to the animal a polypeptide comprising amino acid residues His-Phe-Phe-Lys of hMBP, wherein the polypeptide is about 15 amino acids.

15. A method of tolerizing the immune system of an animal, comprising administering to the animal a polypeptide comprising amino acid residues His-Phe-Phe-Lys of hMBP, joined by chemical means with another epitope of hMBP.

16. A method of tolerizing the immune system of an animal, comprising administering to the animal a polypeptide comprising amino acid residues Lys-Ile-Phe-Lys of hMBP, wherein the polypeptide is about 15 amino acids.

17. A method of tolerizing the immune system of an animal, comprising administering to the animal a polypeptide comprising amino acid residues Lys-Ile-Phe-Lys of hMBP, joined by chemical means with another epitope of hMBP.

18. A method of tolerizing the immune system of an animal, comprising administering to the animal a polypeptide comprising amino acid residues His-Phe-Phe-Lys and Lys-Ile-Phe-Lys of hMBP.

19. A method of treating multiple sclerosis, comprising administering to a patient a polypeptide consisting of nine amino acids substantially homologous with residues 87-99 of human myelin basic protein (hMBP).

20. A method of treating multiple sclerosis, comprising administering to a patient a polypeptide comprising amino acid residues 87-99 of hMBP or residues substantially homologous therewith, wherein the polypeptide is about 15 amino acids.

21. A method of treating multiple sclerosis, comprising administering to a patient a polypeptide comprising amino acid residues 87-99 of hMBP or residues substantially homologous therewith, wherein at least one residue within the region 87-99 is substituted with another amino acid.

22. A method of treating multiple sclerosis, comprising administering to a patient a polypeptide comprising amino acid residues 87-99 of hMBP or residues substantially homologous therewith, wherein at least two residues within the region 87-99 are substituted with other amino acids.

23. A method of treating multiple sclerosis, comprising administering to a patient a polypeptide comprising amino acid residues His-Phe-Phe-Lys of hMBP, wherein the polypeptide is about 15 amino acids.

24. A method of treating multiple sclerosis, comprising administering to a patient a polypeptide comprising amino acid residues His-Phe-Phe-Lys of hMBP, joined by chemical means with another epitope of hMBP.

25. A method of treating multiple sclerosis, comprising administering to a patient a polypeptide comprising amino acid residues Lys-Ile-Phe-Lys of hMBP, wherein the polypeptide is about 15 amino acids.

26. A method of treating multiple sclerosis, comprising administering to a patient a polypeptide comprising amino acid residues Lys-Ile-Phe-Lys of hMBP, joined by chemical means with another epitope of hMBP.

27. A method of treating multiple sclerosis, comprising administering to a patient a polypeptide comprising amino residues His-Phe-Phe-Lys and Lys-Ile-Phe-Lys of hMBP.


SEQUENCE LISTING (52 sequences)

SEQ ID NO: 1 - DNA, 516 bp; single-stranded, linear
  CDS 1..513
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgccaaagcggggctctggcaaggactcacaccacccggcaagaactgctcactatggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctga

SEQ ID NO: 2 - protein, 171 aa
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 3 - protein, 4 aa
GAPS

SEQ ID NO: 4 - protein, 4 aa
GAVG

SEQ ID NO: 5 - protein, 4 aa
EWVS

SEQ ID NO: 6 - protein, 4 aa
KVPT

SEQ ID NO: 7 - protein, 5 aa
GVVLG

SEQ ID NO: 8 - protein, 5 aa
GAVIG

SEQ ID NO: 9 - protein, 4 aa
GILG

SEQ ID NO: 10 - protein, 4 aa
KAAS

SEQ ID NO: 11 - protein, 5 aa
  Modified-site 1: /note= “Where Xaa is Ac-Ala and where Ac is either an alkyl or acyl group”
XSQKR

SEQ ID NO: 12 - protein, 5 aa
KYLAT

SEQ ID NO: 13 - protein, 4 aa
GILD

SEQ ID NO: 14 - protein, 4 aa
RFFG

SEQ ID NO: 15 - protein, 4 aa
HFFK

SEQ ID NO: 16 - protein, 4 aa
KIFK

SEQ ID NO: 17 - protein, 5 aa
  Modified-site 1: /note= “Where Xaa is Ac-Ser and where Ac is either an alkyl or acyl group”
XNKFL

SEQ ID NO: 18 - protein, 4 aa
KFLG

SEQ ID NO: 19 - protein, 4 aa
KLVS

SEQ ID NO: 20 - protein, 4 aa
EYMK

SEQ ID NO: 21 - protein, 4 aa
GLAT

SEQ ID NO: 22 - protein, 5 aa
RVIIS

SEQ ID NO: 23 - protein, 5 aa
KMVVE

SEQ ID NO: 24 - protein, 4 aa
RIYE

SEQ ID NO: 25 - protein, 4 aa
GLLE

SEQ ID NO: 26 - protein, 4 aa
KLIE

SEQ ID NO: 27 - protein, 4 aa
HAFQ

SEQ ID NO: 28 - protein, 4 aa
GAVR

SEQ ID NO: 29 - protein, 4 aa
KWLG

SEQ ID NO: 30 - protein, 4 aa
KFVG

SEQ ID NO: 31 - protein, 4 aa
RMYG

SEQ ID NO: 32 - protein, 4 aa
KLMG

SEQ ID NO: 33 - protein, 4 aa
LVAK

SEQ ID NO: 34 - protein, 4 aa
KIWR

SEQ ID NO: 35 - protein, 5 aa
EWVIK

SEQ ID NO: 36 - protein, 5 aa
KVFID

SEQ ID NO: 37 - protein, 4 aa
KIFT

SEQ ID NO: 38 - protein, 5 aa
KYIAE

SEQ ID NO: 39 - protein, 13 aa
VHFFKNIVTPRTP

SEQ ID NO: 40 - protein, 13 aa
AHFFKNIVTPRTP

SEQ ID NO: 41 - protein, 13 aa
VAFFKNIVTPRTP

SEQ ID NO: 42 - protein, 13 aa
VHAFKNIVTPRTP

SEQ ID NO: 43 - protein, 13 aa
VHFAKNIVTPRTP

SEQ ID NO: 44 - protein, 13 aa
VHFFANIVTPRTP

SEQ ID NO: 45 - protein, 13 aa
VHFFKAIVTPRTP

SEQ ID NO: 46 - protein, 13 aa
VHFFKNAVTPRTP

SEQ ID NO: 47 - protein, 13 aa
VHFFKNIATPRTP

SEQ ID NO: 48 - protein, 13 aa
VHFFKNIVAPRTP

SEQ ID NO: 49 - protein, 13 aa
VHFFKNIVTARTP

SEQ ID NO: 50 - protein, 13 aa
VHFFKNIVTPATP

SEQ ID NO: 51 - protein, 13 aa
VHFFKNIVTPRAP

SEQ ID NO: 52 - protein, 13 aa
VHFFKNIVTPRTA